(19) United States
(12) Patent Application Publication
(10) Pub. No.: US 2024/0087741 A1
(43) Pub. Date: Mar. 14, 2024

(54) CONVERGED MCT AND HOLTER CARDIAC REPORTING

(71) Applicant: Preventice Solutions, Inc., Rochester, MN (US)

(72) Inventors: Mark Alan Pasch, Rochester, MN (US); Michael Thomas Edward McRoberts, Mazeppa, MN (US); Dave H. Gross, Rochester, MN (US)

(21) Appl. No.: 18/139,710

(22) Filed: Apr. 26, 2023

Related U.S. Application Data
(60) Provisional application No. 63/335,614, filed on Apr. 27, 2022.

(51) Int. Cl. G16H 50/20; A61B 5/00; G16H 15/00; G16H 50/30

(52) U.S. Cl. CPC G16H 50/20; A61B 5/0006; G16H 15/00; G16H 50/30

(57) ABSTRACT

A method includes completing, at a server, a mobile cardiac telemetry (MCT) study based on packets of electrocardiogram (ECG) data periodically received by the server. The method further includes completing, at the server, a Holter study based on the packets of ECG data. Completing the Holter study includes generating a report that includes results of the Holter study and the MCT study.

### CROSS REFERENCE TO RELATED APPLICATION

This application claims priority to Provisional Application No. 63/335,614, filed Apr. 27, 2022, all of which are herein incorporated by reference in their entirety.

### TECHNICAL FIELD

The present disclosure relates to devices, methods, and systems for analyzing cardiac activity and cardiac events.

### BACKGROUND

Monitoring devices for collecting biometric data are becoming increasingly common in diagnosing and treating medical conditions in patients. For example, mobile devices can be used to monitor cardiac data in a patient. This cardiac monitoring can empower physicians with valuable information regarding the occurrence and regularity of a variety of heart conditions and irregularities in patients. Cardiac monitoring can be used, for example, to identify abnormal cardiac rhythms, so that critical alerts can be provided to patients, physicians, or other care providers and patients can be treated.

### SUMMARY

In Example 1, a method includes completing, at a server, a mobile cardiac telemetry (MCT) study based on packets of electrocardiogram (ECG) data periodically received by the server. The method further includes completing, at the server, a Holter study based on the packets of ECG data.

In Example 2, the method of Example 1, wherein the completing the MCT study includes automatically detecting cardiac events based on the packets of ECG data.

In Example 3, the method of any of the preceding Examples, wherein the completing the MCT study includes sending a notification about a detected cardiac event to a patient or a physician.

In Example 4, the method of any of the preceding Examples, wherein the completing the Holter study includes generating a package of data, wherein the package of data includes strips of ECG data, metadata associated with the strips of ECG data, and a summary of the automatically detected cardiac events.

In Example 5, the method of Example 4, further including transmitting, to a remote computing system, the package of data and receiving, at the server, a Holter report.

In Example 6, the method of Example 5, wherein the Holter report includes a list of cardiac events detected during the MCT study.

In Example 7, the method of Example 6, wherein the Holter report includes the cardiac events and ECG data associated with the cardiac events.

In Example 8, the method of any of Examples 5-7, wherein the Holter report includes patient-initiated events and ECG data associated with the patient-initiated events.

In Example 9, the method of any of Examples 5-8, further including transmitting executable code to the remote computing system along with the package of data.

In Example 10, the method of any of the preceding Examples, wherein the packets of ECG data include all ECG data generated during the MCT study.

In Example 11, the method of any of the preceding Examples, wherein the completing the Holter study includes generating a final Holter report.

In Example 12, the method of any of the preceding Examples, wherein the completing the Holter study occurs only after the completing the MCT study.

In Example 13, a computer program product comprising instructions to cause one or more processors to carry out the steps of the method of Examples 1-12.

In Example 14, a computer-readable medium having stored thereon the computer program product of Example 13.

In Example 15, a computer comprising the computer-readable medium of Example 14.

In Example 16, a system includes a server with a first processor and a first computer-readable medium having a first set of computer-executable instructions embodied thereon. The first set of instructions are configured to be executed by the first processor to cause the first processor to, as part of an MCT study: detect, using a machine learning model, cardiac events based on packets of ECG data periodically received by the server; determine a severity of the detected events; and based on the severity, cause a notification to be sent to a user. The first set of instructions are configured to be executed by the first processor to cause the first processor to, as part of a Holter study: determine, using the machine learning model, rhythm classifications of events based on the packets of ECG data.

In Example 17, the system of Example 16, wherein the first set of instructions are configured to be executed by the first processor to cause the first processor to, as part of the Holter study: determine, using the machine learning model, beat classifications based on the packets of ECG data.

In Example 18, the system of Example 17, wherein the first set of instructions are configured to be executed by the first processor to cause the first processor to, as part of the Holter study: generate a package of data. The package of data includes strips of the ECG data, the rhythm classifications, the beat classifications, and the detected cardiac events from the MCT study.

In Example 19, the system of Example 18, wherein the detected cardiac events include patient-initiated cardiac events and automatically-detected cardiac events.

In Example 20, the system of Example 18, wherein the first set of instructions are configured to be executed by the first processor to cause the first processor to, as part of the Holter study: transmit, to a remote computing system, the package of data; and receive, at the server, a Holter report generated by the remote computing system.

In Example 21, the system of Example 20, wherein the Holter report includes a list of the detected cardiac events from the MCT study.

In Example 22, the system of Example 20, wherein the Holter report includes strips of ECG data associated with the detected cardiac events from the MCT study.

In Example 23, the system of Example 20, further comprising the remote computing system with, a second processor, and a second computer-readable medium having a second set of computer-executable instructions embodied thereon. The second set of instructions are configured to be executed by the second processor to cause the second processor to: generate the Holter report.

In Example 24, the system of Example 23, wherein the first set of instructions are configured to be executed by the first processor to cause the first processor to: transmit executable code to the remote computing system along with the package of data.

In Example 25, the system of Example 24, wherein the second set of instructions includes the executable code.

In Example 26, the system of Example 16, wherein the first set of instructions are configured to be executed by the first processor to cause the first processor to: complete the MCT study before initiating the Holter study.

In Example 27, the system of Example 16, wherein the severity includes a stable event, a serious event, and a critical event.

In Example 28, the system of Example 16, wherein the machine learning model comprises a deep learning neural network.

In Example 29. the system of Example 16, wherein the ECG data periodically received by the server is generated by a remote ECG monitor attached to a patient.

In Example 30, a method includes completing, at a server, a MCT study based on packets of ECG data periodically received by the server. Completing the MCT study includes automatically detecting, using a machine learning model, cardiac events based on packets of ECG data. The method further includes completing, at the server, a Holter study based on the packets of ECG data. Completing the Holter study includes determining, using the machine learning model, rhythm classifications of events based on packets of ECG data.

In Example 31, the method of Example 30, wherein completing the MCT study includes sending a notification about a detected cardiac event to a patient or a physician.

In Example 32, the method of Example 30, wherein completing the Holter study includes generating a package of data. The package of data includes strips of ECG data, the rhythm classifications, and a summary of the automatically detected cardiac events from the MCT study.

In Example 33, the method of Example 32, further including: transmitting, to a remote computing system, the package of data; and receiving, at the server, a Holter report.

In Example 34, the method of Example 33, wherein the Holter report includes a list of the automatically detected cardiac events from the MCT study and ECG data associated with the automatically detected cardiac events.

In Example 35, the method of Example 30, wherein the completing the Holter study occurs only after the completing the MCT study.

While multiple instances are disclosed, still other instances of the present disclosure will become apparent to those skilled in the art from the following detailed description, which shows and describes illustrative instances of the disclosure. Accordingly, the drawings and detailed description are to be regarded as illustrative in nature and not restrictive.

### BRIEF DESCRIPTION OF THE DRAWINGS

FIG. 1 shows a cardiac monitoring system, in accordance with certain instances of the present disclosure.

FIG. 2 shows a server, remote computer, and user interface, in accordance with certain instances of the present disclosure.

FIG. 3 shows an example MCT end of service report, in accordance with certain instances of the present disclosure.

FIG. 4 shows a view of a report build page, in accordance with certain instances of the present disclosure.

FIG. 5 shows a view of a report simulation page, in accordance with certain instances of the present disclosure.

FIG. 6 shows a flow diagram depicting an illustrative method for combining results from MCT studies and Holter studies, in accordance with instances of the disclosure.

FIG. 7 is a block diagram depicting an illustrative computing device, in accordance with instances of the disclosure.

While the disclosed subject matter is amenable to various modifications and alternative forms, specific instances have been shown by way of example in the drawings and are described in detail below. The intention, however, is not to limit the disclosure to the particular instances described. On the contrary, the disclosure is intended to cover all modifications, equivalents, and alternatives falling within the scope of the disclosure as defined by the appended claims.

### DETAILED DESCRIPTION

The present disclosure relates to devices, methods, and systems for facilitating analysis of cardiac activity and cardiac events (e.g., abnormal cardiac rhythms or other issues). Electrocardiogram (ECG) data of a patient can be used to monitor a patient and identify whether the patient has experienced a cardiac event and what type of cardiac event occurred.

One type of ECG-based study is called mobile cardiac telemetry monitoring, which is sometimes referred to as MCOT monitoring or MCT monitoring. Using MCT monitoring, a patient's cardiac activity is monitored in near real time, and the patient's physician is notified if the cardiac activity meets certain criteria established by the physician. At the end of the MCT monitoring period (or at various periods during the period), an end of service report can be created to summarize the cardiac events detected during MCT monitoring period.

Another type of ECG-based study is referred to as ambulatory cardiac rhythm monitoring, which is sometimes referred to as Holter monitoring. Holter monitoring is a retrospective analysis of longer study (e.g., 24- to 48-hour or sometimes longer study done on ambulatory patients). At the end of the Holter monitoring period, a final report can be generated to provide a summary of the monitored cardiac activity and provide representative examples of the detected rhythms and beats. The final Holter report includes several more details than end of service reports generated for MCT studies. However, Holter monitoring does not necessarily involve near-real-time physician notifications.

Instances of the present disclosure are accordingly directed to systems, methods, and devices for combining aspects of MCT monitoring and Holter monitoring.

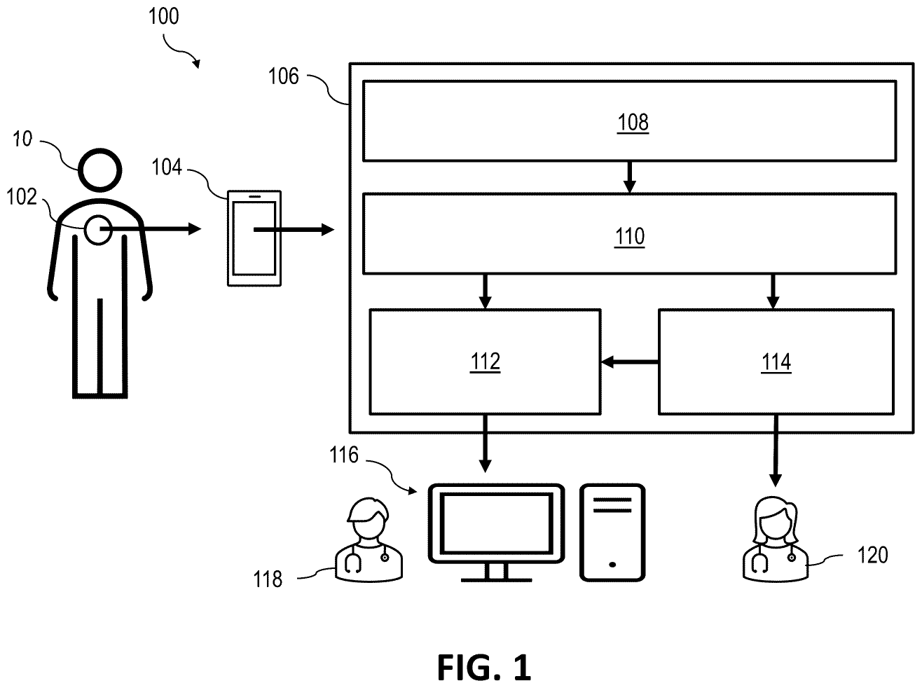

FIG. 1 illustrates a patient 10 and an example system 100. The system 100 includes a monitor 102 attached to the patient 10 to detect cardiac activity of the patient 10. The monitor 102 may produce electric signals that represent the cardiac activity in the patient 10. For example, the monitor 102 may detect the patient's heart beating (e.g., using infrared sensors, electrodes) and convert the detected heartbeat into electric signals representing ECG data. The monitor 102 communicates the ECG data to a mobile device 104 (e.g., a mobile phone).

The mobile device 104 may include a program (e.g., mobile phone application) that receives, processes, and analyzes the ECG data. For example, the program may analyze the ECG data and detect or flag cardiac events (e.g., periods of irregular cardiac activity) contained within the ECG data. As noted above, because ECG data may be getting continuously generated, the amount of ECG data can be overwhelming to store and process locally on the mobile device 104. As such, the mobile device 104 can periodically transmit chunks of the ECG data (e.g., in 1- to 20-minute intervals) to another device or system, which can process, append together, and archive the chunks of the ECG data and metadata (e.g., time, duration, detected/flagged cardiac events) associated with the chunks of ECG data. In certain instances, the monitor 102 may be programmed to transmit the ECG data directly to the other device or system without utilizing the mobile device 104.

In certain instances, the monitor 102 and/or the mobile device 104 includes a button or touch-screen icon that allows the patient 10 to initiate an event. Such an indication can be recorded and communicated to the other device or system. In certain instances, patient-initiated events are timestamped and cause the monitor 102 and/or the mobile device 104 to send a package of ECG data surrounding the patient-initiated event to a server.

### Cardiac Event Server

In the example shown in FIG. 1, the mobile device 104 transmits the ECG data (and associated metadata, if any) to a cardiac event server 106 (hereinafter “the server 106” for brevity). The server 106 includes multiple platforms, layers, or modules that work together to process and analyze the ECG data such that cardiac events can be detected, filtered, prioritized, and ultimately reported to a patient's physician for analysis and treatment. In the example of FIG. 1, the server 106 includes one or more machine learning models 108 (e.g., types of deep neural networks), a cardiac event router 110, a Holter platform 112, and an MCT platform 114.

Although only one server 106 is shown in FIG. 1, the server 106 can include multiple separate physical servers, and the various platforms/modules/layers can be distributed among the multiple servers. Each of the platforms/modules/layers can represent separate programs, applications, and/or blocks of code where the output of one of the platforms/modules/layers is an input to another of the platforms/modules/layers. Each platform/module/layer can use application programming interfaces to communicate between or among the other platforms/modules/layers as well as systems and devices external to the server 106.

The server 106 applies the machine learning model 108 to the ECG data to classify cardiac activity of the patient 10. For example, the machine learning model 108 may compare the ECG data to labeled ECG data to determine which labeled ECG data the ECG data most closely resembles. The labeled ECG data may identify a particular cardiac event, including but not limited to ventricular tachycardia, bradycardia, atrial fibrillation, pause, normal sinus rhythm, or artifact/noise. Further, the labeled ECG data may identify beat classifications such as normal, ventricular, and supraventricular.

In certain embodiments, the machine learning model 108 includes two paths, where the first path is a deep convolutional neural network, and the second path is a deep fully-connected neural network. The deep convolutional neural network receives one or more sets of beats (e.g., beat trains with 3-10 beats) which are processed through a series of layers in the deep convolutional neural network. The series of layers can include a convolution layer to perform convolution on time series data in the beat trains, a batch normalization layer to normalize the output from the convolution layer (e.g., centering the results around an origin), and a non-linear activation function layer to receive the normalized values from the batch normalization layer. The beat trains then pass through a repeating set of layers such as another convolution layer, a batch normalization layer, a non-linear activation function layer. This set of layers can be repeated multiple times.

The deep fully connected neural network receives RR-interval data (e.g., time intervals between adjacent beats) and processes the RR-interval data through a series of layers: a fully connected layer, a non-linear activation function layer, another fully connected layer, another non-linear activation function layer, and a regularization layer. The output from the two paths is then provided to the fully connected layer. The resulting values are passed through a fully connected layer and a softmax layer to produce probability distributions for the classes of beats.

If the machine learning model 108 determines that the ECG data most closely resembles a labeled ECG data associated with a cardiac event, then the machine learning model 108 may determine that the patient 10 has experienced that cardiac event. Additionally, the machine learning model 108 may measure or determine certain characteristics of the cardiac activity of the patient 10 based on the ECG data. For example, the machine learning model 108 may determine a heart rate, a duration, or a beat count of the patient 10 during the cardiac event based on the ECG data. The server 106 stores the cardiac event (and associated metadata such as information like beat classification, heart rate, duration, beat count, etc.) in a database for storage. Subsequently, the server 106 may retrieve the cardiac event and associated information from the database.

In certain instances, the mobile device 104 (or monitor 102) may initially classify a cardiac event based on the ECG data. The server 106 may then re-classify or confirm the cardiac event using the machine learning model 108. Doing so allows for a more computationally-expensive analysis of the ECG data to be performed using the computing resources of the server 106, rather than the limited resources of the mobile device 104.

In certain instances, once the ECG data is processed by the machine learning model 108, the ECG data is made available for the Holter platform 112 and/or the MCT platform 114. As will be described in more detail below, the Holter platform 112 and the MCT platform 114 can be accessed by respective remote computers 116 (e.g., client device such as a laptop, mobile phone, desktop computer, and the like) by a user at a clinic or lab 118.

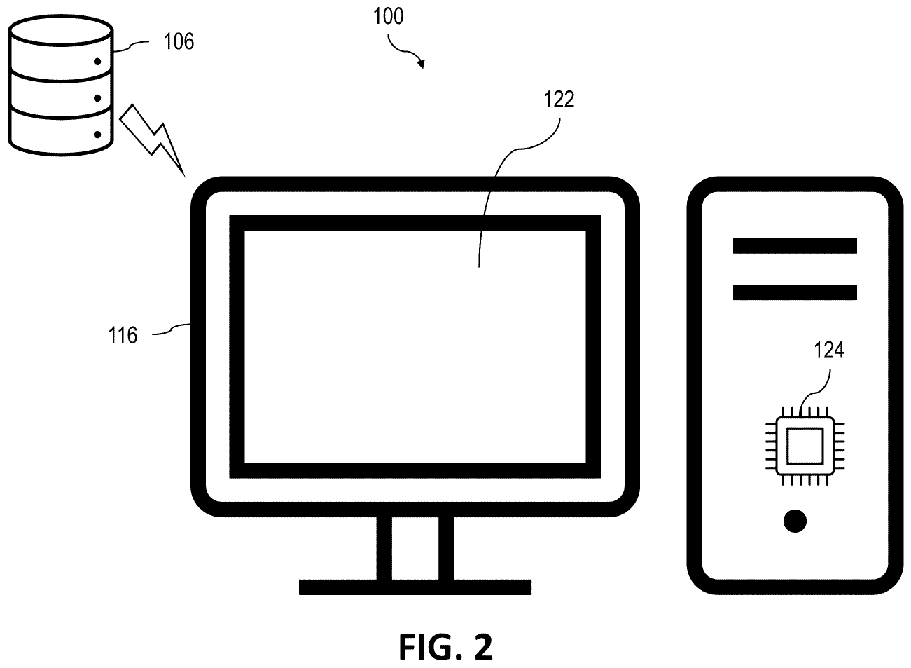

FIG. 2 shows the server 106 communicatively coupled (e.g., via a network) to the remote computer 116. In the example of FIG. 2, the remote computer 116 includes a monitor showing a user interface 122 (hereinafter “the UI 122” for brevity) that displays features of the Holter platform 112 and/or MCT platform 114 hosted by the server 106. The UI 122 includes multiple pages or screens for tracking and facilitating analysis of patient ECG data.

Referring back to FIG. 1, in certain instances, the cardiac event router 110 is used to determine what platform will further process the ECG data. That determination can be based on the classification associated with the cardiac event. For example, if the identified cardiac event is severe, the cardiac event router 110 can flag or send the ECG data, etc., to the MCT platform 114. The MCT platform 114 can be programmed to send notifications (along with relevant ECG data and associated metadata) immediately to the patient's physician/care group (e.g., to their computer system, e-mail, mobile phone application) and/or to the patient 10 (e.g., to their computer system, e-mail, mobile phone application).

In other instances, the server 106 does not include the cardiac event router 110. Instead, the server 106 may be programmed to first route ECG data (and metadata) from the machine learning model 108 to the MCT platform 114 and then to the Holter platform 112 at the end of an MCT study.

### MCT Platform

During an MCT study, the server 106 periodically receives chunks of ECG data, which are processed by the machine learning model 108. The machine learning model 108 is programmed to automatically detect that a cardiac event has occurred and to associate the detected cardiac event with an initial event classification (e.g., as an atrial fibrillation event, ventricular tachycardia event, flutter event, and the like). Further, the machine learning model 108 can determine an initial acuity or severity for each detected event. For example, each detected event can be determined to be a stable event, a serious event, or a critical event. In addition, the machine learning model 108 may associate each individual beat with a beat classification.

The determined events and classifications can be analyzed by the MCT platform 114 to determine whether and how the detected events should be further processed and escalated. Once a cardiac event is determined to be a critical event, the MCT platform 114 can send a notification to a patient and/or their physician/care group. In certain instances, before the notification is sent, a technician (e.g., an ECG technician) intercepts the notification and confirms whether the cardiac event is a critical event. Additionally, during an MCT study, technicians may review each detected event and determine whether the associated acuity and event classification are correct. The technician can prioritize cardiac events initially designated to be critical events, followed by serious events, and then followed by stable events.

As noted above, at the end of the MCT monitoring period, an end of service (EOS) report can be created to summarize the cardiac events detected during MCT monitoring period.

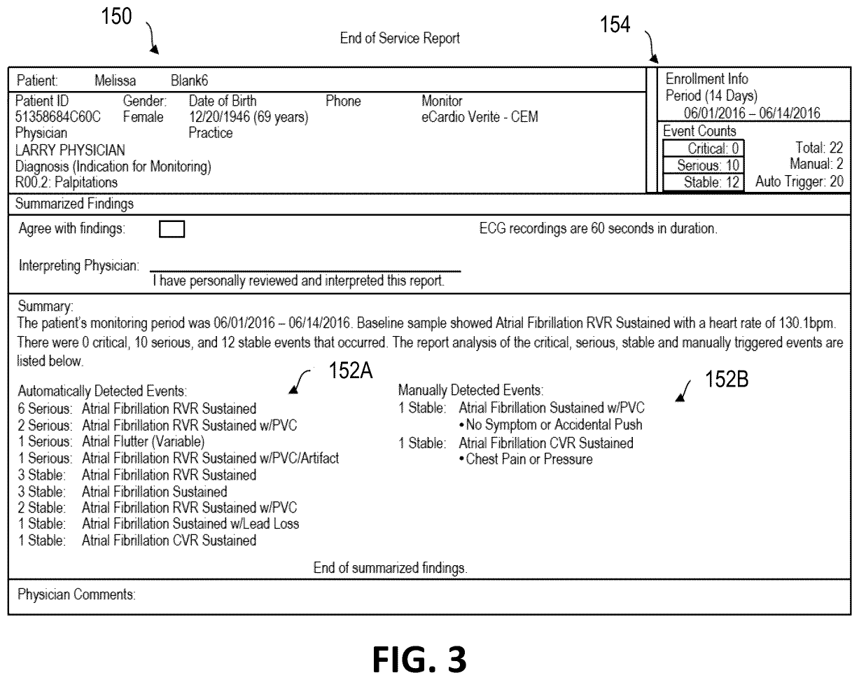

FIG. 3 shows an example EOS report 150. The EOS report 150 includes a list of cardiac events detected during the MCT study. In the example of FIG. 3, the cardiac events are separated into two lists. The first list 152A includes the cardiac events that were automatically detected by the machine learning model 108, and the second list 1526 includes the cardiac events that were patient-initiated. The listed events can be ordered by their severity. For each category of events in the lists 152A and 152B, the categories can indicate the severity of the events, a description of the event category, and the number of events that occurred during the MCT study. The EOS report 150 can also include a high-level summary 154 of the number of events—by severity—that occurred during the MCT study.

As can be seen in FIG. 3, the EOS report 150 lacks information such as the underlying ECG data, the types of beats, RR intervals, among other things that may be useful for the physician. To help generate such additional information, the underlying ECG data from the MCT study can be processed by the Holter platform 112 to initiate a Holter study. For example, the underlying ECG data can be processed again by the machine learning model 108 and then structured to be sent to the remote computing system 116. In certain instances, reprocessing the ECG data occurs only after the MCT study has been completed. The MCT study can be completed upon receiving ECG data for a predetermined period of time (e.g., 14 days, 30 days).

### Holter Platform

The Holter platform 112 can be a software-as-a-service (SaaS) platform hosted by the server 106. To access the Holter platform 112, a user (e.g., a technician) interacts with the UI 122 to log into the Holter platform 112 via a web browser such that the user can use and interact with the Holter platform 112. When the user at the clinic or lab 118 is ready to analyze ECG data of a patient, the user can select a patient's profile through the UI 122.

The server 106 (e.g., via programming associated with the Holter platform 112) can start a process for sending data (e.g., packages of data) to the remote computer 116. This data includes the ECG data (e.g., strips of ECG data) and metadata associated with the ECG data. The package of data can also include data structures representing the cardiac events detected during the MCT study as well as metadata associated with each detected event such as the severity and event classification.

As noted above, once the ECG data from the monitored patients has been collected, the machine learning model 108 may determine certain characteristics of the cardiac activity of the patient 10 based on the ECG data, including estimating that a cardiac event has occurred and associating or generating metadata for the determined events. The metadata can include information about the patient 10, a heart rate of the patient 10 during the cardiac event, a duration of the cardiac event, a beat count of the cardiac event, a confidence level of the machine learning model's identification of the cardiac event, and/or a beat classification (e.g., normal, ventricular, supraventricular, unclassified).

Accessing, processing, and displaying one or more days' worth of ECG data and metadata can consume a large amount of computing resources, network bandwidth resources, and human resources. To help alleviate burdens on these resources, the server 106 (e.g., via the Holter platform 112) can selectively transmit packages of ECG data and metadata to the remote computer 116.

The initial packages of data can include: (1) short strips (e.g., 60-second strips) of ECG data surrounding detected cardiac events, (2) metadata associated with the strips, and (3) executable code (e.g., JavaScript code). In certain instances, only the ECG data associated with highest priority cardiac events are initially transferred. After the initial packages of data are transmitted from the server to the remote computer 116, additional packages of data can be transmitted in response to selections made by the user in the U 1122.

### Holter Report Build Page

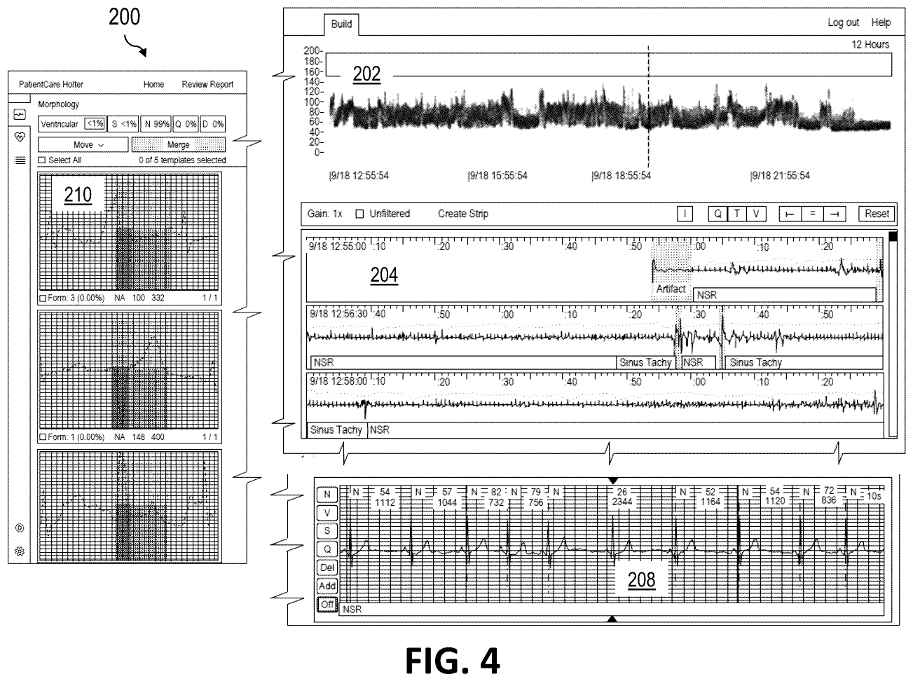

With these initial packages of data, the user has access (via the remote computer 116 and the UI 122) to a Holter report build page 200 shown in FIG. 4. The Holter report build page 200 facilitates analysis of cardiac events and metadata associated with the cardiac events. The pages are generated at the remote computer 116 based on the packages of data and they are selectively displayed via the UI 122. As will be described in more detail below, as the user interacts with the Holter report build page 200, the metadata is updated in real time at the remote computer 116.

FIG. 4 shows a screenshot of the Holter report build page 200. The Holter report build page 200 is used by a user to review and analyze a patient's ECG data and metadata. The Holter report build page 200 includes multiple windows for displaying data, plots, icons, links, markers, indicators, and the like.

Window 202 displays a heart rate plot of multiple days' worth of ECG data. This window 202 provides an initial visual insight into which periods of time appear to contain abnormal heart rate activity. In the example of FIG. 4, the window 202 displays four days of ECG data, although shorter or longer time periods could be displayed by default or changed by the user.

Window 204 allows the user to view a shorter plot of ECG data. For example, the window 204 may display ECG data associated with a detected cardiac event along with ECG data preceding and following the detected event. This window 204 provides visual insight into the onset of a detected event and whether the detected event is perhaps an artifact, follow-on event, etc. As the user scrolls through the window 204, the window 202 can display an indicator (e.g., a vertical line) showing the location of the ECG data of window 204 within the heart rate plot of window 202.

Window 208 shows a plot of ECG data (e.g., approximately 10 beats) that is shorter than the plots of windows 202 and 204. Window 208 displays a closer-up view of a portion of the ECG data of windows 202 and 204. The user can use window 204 to select which shorter set of ECG data is displayed in the window 208. Each of the windows 202, 204, and 208 can include markers, indicators, icons, etc., to visually note the location of detected cardiac events within the strips of ECG data.

### Combined MCT and Holter Studies

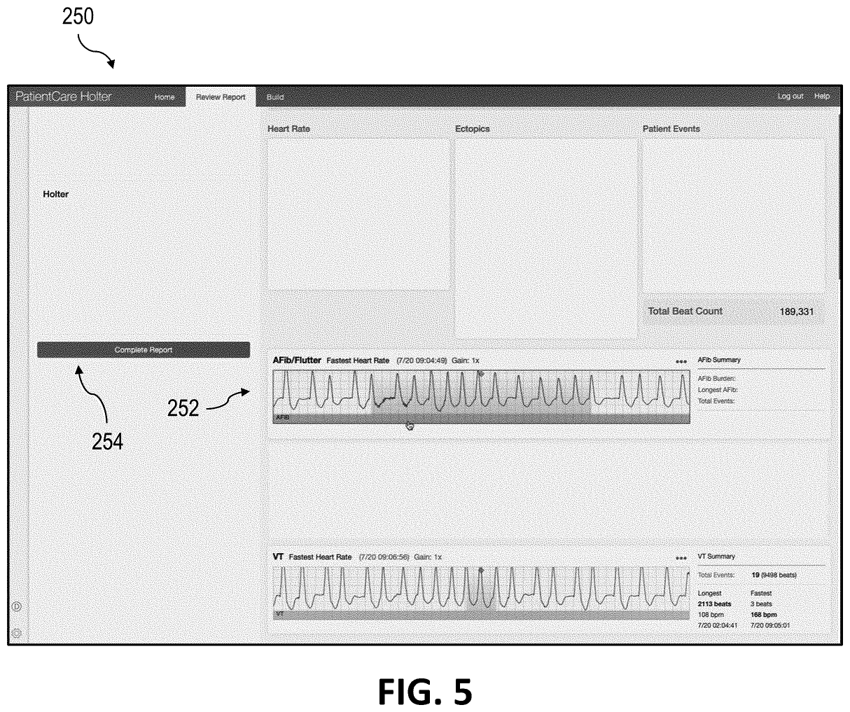

FIG. 5 shows a screenshot of a report simulation page 250. The initial ECG data, metadata, and strips of ECG data shown in the report simulation page 250 can be generated by the Holter report platform 112. That is, when the user initially selects the report simulation page 250, the user is viewing an initial simulated report that was automatically generated by the Holter report platform 112.

In addition, the report simulation page 250 can include the list of cardiac events detected during the MCT study, including the severity and rhythm classification of each detected cardiac event as well as any recorded symptoms. In certain instances, the report simulation page 250 also includes strips of ECG data (e.g., the ECG waveform) associated with the cardiac events detected during the MCT study. By incorporating the results of the MCT study into the Holter study, the details of the detected cardiac events found in the MCT study can be included in the final Holter report (which is discussed in more detail below).

The simulated report shown in the report simulation page 250 mimics the formatting and data that would ultimately be included in a finalized Holter report sent to the patient's physician. Example finalized reports include Holter reports (e.g., short-term or long-term) and cardiac event reports, and the reports can be in the form of a pdf file or other file type.

Because the simulated report displayed on the report simulation page 250 mimics the final report, the simulated report allows the user to see a preview of the final report without needing to spend the time generating an actual finalized report, which can take 10-20 minutes. Thus, the report simulation page 250 saves the user time from having to create multiple reports before settling on a final version that is then sent to the patient's physician.

In addition to information from the MCT study, the report simulation page 250 includes a baseline summary of patient information generated from the ECG data. For example, as shown in FIG. 5, the report simulation page 250 includes a summary of heart rate information, ectopics, and events—each of which may have been initially generated by the machine learning model 108. For a given type of event, the report simulation page 250 includes one or more example strips of ECG data 252.

The report simulation page 250 can be automatically updated in real-time as the user makes changes in the Holter report build page 200. For example, if the user recharacterizes beats with a different type of cardiac event, the report simulation page 250 will be updated to reflect the change. This updating of data may include automatically changing the order of cardiac events on the report simulation page 250, changing which strip of ECG data is representative of a given cardiac event, and/or updating the baseline summary of patient information generated from the ECG data, among other things. Further, as the user selects representative events on the Holter report build page 200, those events are automatically added to the report simulation page 250.

To save processing and network resources and to allow these changes to metadata to occur in real-time, the calculations and changes to the cardiac event classifications and the automatic updates to the beat classifications can be carried out locally on the remote computer 116—as opposed to sending data back and forth between the server 106 and the remote computer 116. For example, the reclassifications can be carried out using cache memory 124 (shown in FIG. 2) and processing capabilities (e.g., one or more microprocessors) of the remote computer 116. To enable local processing and updating, the Holter platform 112 can send the remote computer 116 code to execute locally. This code uses (or operates on) the outputs of the machine learning model 108 such as the beat classifications and rhythm classifications (as opposed to the underlying or raw ECG data), which reduces the computational resources needed to process the changes made by user locally at the remote computer 116. In certain embodiments, this code is executed by an internet browser operating on the remote computer 116.

Once the user is satisfied with the simulated report, the user can select the “Complete Report” button 254 on the report simulation page 250. This selection will start the process of generating a final report to be sent to the patient's physician. In certain instances, the server 106 generates the final report.

In certain instances, once the report is built and complete, the remote computer 116 can send the report and any changes to the metadata (e.g., the subsequent beat classifications and the subsequent rhythm classifications) to the server 106 and its database. The server 106 can then replace the metadata initially created by the machine learning model (and saved to the database) with the metadata generated by the remote computer while the user was reviewing and editing the metadata. As such, if the ECG and metadata need to be accessed again, the server's database has the most recent version of the metadata. Further, the machine learning model 108 may be further trained on the metadata generated by the user at the remote computer.

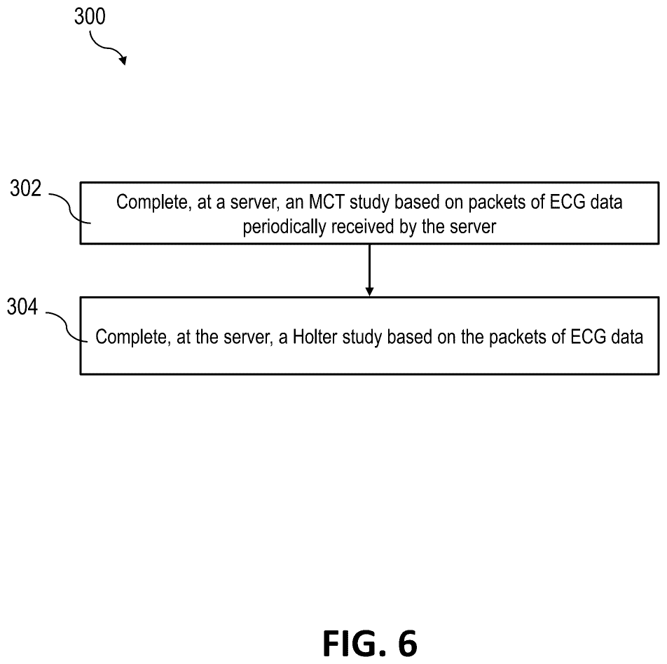

FIG. 6 shows a block diagram of a method 400 for combining results from MCT studies and Holter studies. The method 400 completing, at a server, an MCT study based on packets of ECG data periodically received by the server (block 402 in FIG. 6). The method 400 further includes completing, at the server, a Holter study based on the packets of ECG data (block 404 in FIG. 6).

### Computing Devices and Systems

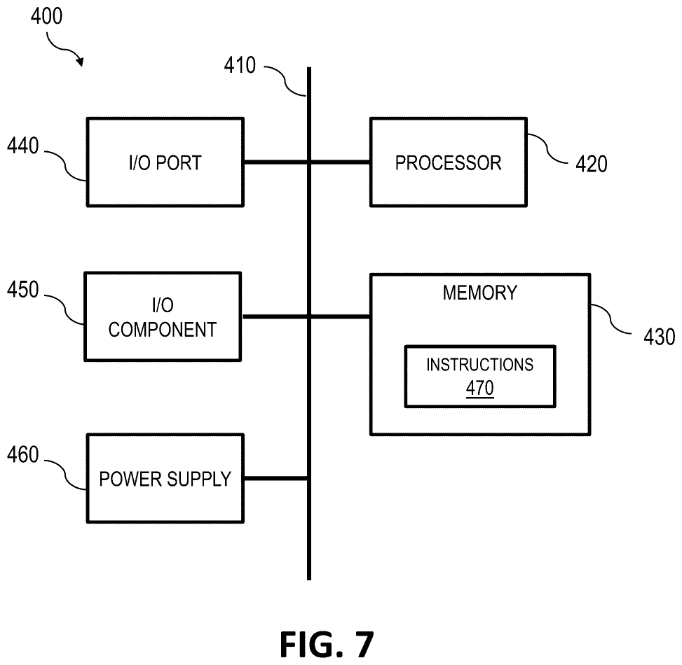

FIG. 7 is a block diagram depicting an illustrative computing device 400, in accordance with instances of the disclosure. The computing device 400 may include any type of computing device suitable for implementing aspects of instances of the disclosed subject matter. Examples of computing devices include specialized computing devices or general-purpose computing devices such as workstations, servers, laptops, desktops, tablet computers, hand-held devices, smartphones, general-purpose graphics processing units (GPGPUs), and the like. Each of the various components shown and described in the Figures can contain their own dedicated set of computing device components shown in FIG. 7 and described below. For example, the mobile device 104, the server 106, and the remote computer 116 can each include their own set of components shown in FIG. 7 and described below.

In instances, the computing device 400 includes a bus 410 that, directly and/or indirectly, couples one or more of the following devices: a processor 420, a memory 430, an input/output (I/O) port 440, an I/O component 450, and a power supply 460. Any number of additional components, different components, and/or combinations of components may also be included in the computing device 400.

The bus 410 represents what may be one or more busses (such as, for example, an address bus, data bus, or combination thereof). Similarly, in instances, the computing device 400 may include a number of processors 420, a number of memory components 430, a number of I/O ports 440, a number of I/O components 450, and/or a number of power supplies 460. Additionally, any number of these components, or combinations thereof, may be distributed and/or duplicated across a number of computing devices.

In instances, the memory 430 includes computer-readable media in the form of volatile and/or nonvolatile memory and may be removable, nonremovable, or a combination thereof. Media examples include random access memory (RAM); read only memory (ROM); electronically erasable programmable read only memory (EEPROM); flash memory; optical or holographic media; magnetic cassettes, magnetic tape, magnetic disk storage or other magnetic storage devices; data transmissions; and/or any other medium that can be used to store information and can be accessed by a computing device. In instances, the memory 430 stores computer-executable instructions 470 for causing the processor 420 to implement aspects of instances of components discussed herein and/or to perform aspects of instances of methods and procedures discussed herein. The memory 430 can comprise a non-transitory computer readable medium storing the computer-executable instructions 470.

The computer-executable instructions 470 may include, for example, computer code, machine-useable instructions, and the like such as, for example, program components capable of being executed by one or more processors 420 (e.g., microprocessors) associated with the computing device 400. Program components may be programmed using any number of different programming environments, including various languages, development kits, frameworks, and/or the like. Some or all of the functionality contemplated herein may also, or alternatively, be implemented in hardware and/or firmware.

According to instances, for example, the instructions 470 may be configured to be executed by the processor 420 and, upon execution, to cause the processor 420 to perform certain processes. In certain instances, the processor 420, memory 430, and instructions 470 are part of a controller such as an application specific integrated circuit (ASIC), field-programmable gate array (FPGA), and/or the like. Such devices can be used to carry out the functions and steps described herein.

The I/O component 450 may include a presentation component configured to present information to a user such as, for example, a display device, a speaker, a printing device, and/or the like, and/or an input component such as, for example, a microphone, a joystick, a satellite dish, a scanner, a printer, a wireless device, a keyboard, a pen, a voice input device, a touch input device, a touch-screen device, an interactive display device, a mouse, and/or the like.

The devices and systems described herein can be communicatively coupled via a network, which may include a local area network (LAN), a wide area network (WAN), a cellular data network, via the internet using an internet service provider, and the like.

Aspects of the present disclosure are described with reference to flowchart illustrations and/or block diagrams of methods, devices, systems and computer program products. It will be understood that each block of the flowchart illustrations and/or block diagrams, and combinations of blocks in the flowchart illustrations and/or block diagrams, can be implemented by computer program instructions.

Various modifications and additions can be made to the exemplary instances discussed without departing from the scope of the disclosed subject matter. For example, while the instances described above refer to particular features, the scope of this disclosure also includes instances having different combinations of features and instances that do not include all of the described features. Accordingly, the scope of the disclosed subject matter is intended to embrace all such alternatives, modifications, and variations as fall within the scope of the claims, together with all equivalents thereof.

We claim:

1. A system comprising:
a server comprising: a first processor and a first computer-readable medium having a first set of computer-executable instructions embodied thereon, the first set of instructions configured to be executed by the first processor to cause the first processor to:
as part of a mobile cardiac telemetry (MCT) study:
detect, using a machine learning model, cardiac events based on packets of electrocardiogram (ECG) data periodically received by the server,
determine a severity of the detected events, and
based on the severity, cause a notification to be sent to a user; and
as part of a Holter study:
determine, using the machine learning model, rhythm classifications of events based on the packets of ECG data.

2. The system of claim 1, wherein the first set of instructions are configured to be executed by the first processor to cause the first processor to, as part of the Holter study:
determine, using the machine learning model, beat classifications based on the packets of ECG data.

3. The system of claim 2, wherein the first set of instructions are configured to be executed by the first processor to cause the first processor to, as part of the Holter study:
generate a package of data, wherein the package of data includes strips of the ECG data, the rhythm classifications, the beat classifications, and the detected cardiac events from the MCT study.

4. The system of claim 3, wherein the detected cardiac events include patient-initiated cardiac events and automatically-detected cardiac events.

5. The system of claim 3, wherein the first set of instructions are configured to be executed by the first processor to cause the first processor to, as part of the Holter study:
transmit, to a remote computing system, the package of data, and
receive, at the server, a Holter report generated by the remote computing system.

6. The system of claim 5, wherein the Holter report includes a list of the detected cardiac events from the MCT study.

7. The system of claim 5, wherein the Holter report includes strips of ECG data associated with the detected cardiac events from the MCT study.

8. The system of claim 5, further comprising:
the remote computing system comprising: a second processor, and a second computer-readable medium having a second set of computer-executable instructions embodied thereon, the second set of instructions configured to be executed by the second processor to cause the second processor to:
generate the Holter report.

9. The system of claim 8, wherein the first set of instructions are configured to be executed by the first processor to cause the first processor to:
transmit executable code to the remote computing system along with the package of data.

10. The system of claim 9, wherein the second set of instructions includes the executable code.

11. The system of claim 1, wherein the first set of instructions are configured to be executed by the first processor to cause the first processor to: complete the MCT study before initiating the Holter study.

12. The system of claim 1, wherein the severity comprises a stable event, a serious event, and a critical event.

13. The system of claim 1, wherein the machine learning model comprises a deep learning neural network.

14. The system of claim 1, wherein the ECG data periodically received by the server is generated by a remote ECG monitor attached to a patient.

15. A method comprising:
completing, at a server, a mobile cardiac telemetry (MCT) study based on packets of electrocardiogram (ECG) data periodically received by the server, wherein the completing the MCT study includes automatically detecting, using a machine learning model, cardiac events based on packets of ECG data; and
completing, at the server, a Holter study based on the packets of ECG data, wherein the completing the Holter study includes determining, using the machine learning model, rhythm classifications of events based on packets of ECG data.

16. The method of claim 15, wherein the completing the MCT study includes sending a notification about a detected cardiac event to a patient or a physician.

17. The method of claim 15, wherein the completing the Holter study includes generating a package of data, wherein the package of data includes strips of ECG data, the rhythm classifications, and a summary of the automatically detected cardiac events from the MCT study.

18. The method of claim 17, further comprising:
transmitting, to a remote computing system, the package of data; and
receiving, at the server, a Holter report.

19. The method of claim 18, wherein the Holter report includes a list of the automatically detected cardiac events from the MCT study and ECG data associated with the automatically detected cardiac events.

20. The method of claim 15, wherein the completing the Holter study occurs only after the completing the MCT study.
